(19) United States
(12) Patent Application Publication
(10) Pub. No.: US 2022/0254499 A1
(43) Pub. Date: Aug. 11, 2022

(54) Pre-Emptive Asthma Risk Notifications Based on Medicament Device Monitoring

(71) Applicant: Reciprocal Labs Corporation (d/b/a Propeller Health), Madison, WI (US)

(72) Inventors: Meredith Ann Barrett, Redwood City, CA (US); Mike Lohmeier, Sun Prairie, WI (US); Christopher Hogg, San Francisco, CA (US); John David Van Sickle, Oregon, WI (US); Nicholas John Hirons, Madison, WI (US)

(21) Appl. No.: 17/628,877

(22) PCT Filed: Jul. 23, 2020
(86) PCT No.: PCT/US2020/043289

§ 371 Date: Jan. 20, 2022

Related U.S. Application Data
(60) Provisional application No. 62/879,284, filed on Jul. 26, 2019.

(51) Int. Cl. G16H 50/30; G16H 10/60; G16H 20/13

(52) U.S. Cl. CPC G16H 50/30; G16H 10/60; A61B 2560/0252; A61B 2560/0242; G16H 20/13

(57) ABSTRACT

An asthma analytics system provides asthma risk notifications in advance of predicted rescue usage events in order to help effect behavior changes in a patient to prevent those events from occurring. Rescue medication events, changes in environmental conditions, and other contextually relevant information are detected by sensors associated with the patient's medicament device/s and are collected from other sources, respectively, to provide a basis to determine a patient's risk score. This data is analyzed to determine the severity of the patient's risk for an asthma event and is used to send notifications accordingly.

### BACKGROUND

### Field of Art

The disclosure relates generally to methods of improving treatment for patients who use inhalers, and more specifically to determining a patient's risk of asthma-related rescue events.

### Description of the Related Art

Asthma remains a significant and costly public health problem. Worldwide, the World Health Organization estimates the population with asthma may be 300 million, and predicts that it will rise to 400 million by 2025. In the United States, asthma affects 1 in 12 individuals and prevalence is on the rise, leading to more than $56 billion per year in health care utilization costs.

Despite the development of new medications, rates of hospitalizations and emergency room visits have not declined. Each year in the United States, the disease causes approximately 2 million emergency department visits, 500,000 hospitalizations, and 5,000 deaths. In addition, asthma is responsible for an estimated 15 million missed days of school, and 12 million days of work. Total annual costs to U.S. health insurers and employers are greater than $18 billion.

The majority of these exacerbations could be prevented with currently available treatments. However, only 1 in 5 asthmatics has the disease under control. Newly revised national guidelines urge doctors to more closely monitor whether treatment is controlling everyday symptoms and improving quality of life. Physicians, however, have few available tools to assess how well their patients are doing day-to-day. An increasing number of physicians have begun to use periodic, written questionnaires (such as the Asthma Control Test) to monitor patients and determine their level of control. These instruments require patients to accurately recall and report the frequency of symptoms, inhaler usage, and activity level and restriction over some period of time (usually two to four weeks). As a result, these questionnaires are subject to error introduced by biases (recall), different interpretations of symptoms, and behaviors (non-adherence), and only provide information at the time they are used.

More generally, medicament devices such as inhalers allow patients to manage respiratory symptoms such as constricted airflow. Many respiratory disease patients, such as sufferers of asthma, chronic obstructive pulmonary disease (COPD), and cystic fibrosis, have symptoms that are related to environmental triggers and factors such as air quality, weather, land use, and the like. A patient being aware of which environmental triggers and factors affect their symptoms allows the patient to better manage their symptoms and reduce the chances for needing emergency medical care. However, a particular patient or group of patients may have sensitivities to multiple triggers and factors. Knowing which of dozens, hundreds, or more triggers and factors a patient is sensitive to and monitoring those triggers and factors for use in managing symptoms is a complex task and not currently feasible for many patients and providers.

### SUMMARY

An asthma analytics system is a unified platform for treating, monitoring, and managing rescue events resulting from asthma. The asthma analytics system tracks asthma rescue medication events by receiving event notifications from a sensor attached to a medicament device (e.g., inhaler) used by a patient who has authorized the asthma analytics system to help manage their asthma. The sensor, when attached to or incorporated in a metered dose inhaler or other medicament device, records the geographical location, time, and date of the rescue usage event, and communicates that information to the asthma analytics system. The asthma analytics system analyzes the received events (both the most recent and previously received events) in real-time or near real-time, and delivers a risk assessment and information to guide and manage the disease in the form of a notification to patients and the healthcare providers.

A patient-specific risk score is determined using a combination of parameters including a patient's medical history, a patient's current situation on a day-to-day basis, and environmental conditions relating to atmospheric and weather conditions. The relationship between these parameters and risk assessment generated for the patient is embodied in a machine learned model. The model, and system more generally, is capable of receiving input values for the parameters and predicting a number of medication puffs a patient will take daily. The system may further determine a patient-specific risk score based on the prediction and categorize the risk score to provide a risk assessment with accurate and medically relevant treatment options to mitigate the risk.

By ingesting information about the timing, frequency, and location of the usage of the medication along with other contextually relevant parameter information, the asthma analytics system helps prevent the occurrence of future asthma rescue usage events by suggesting immediately applicable changes in behavior or environment in advance of those predicted events. This facilitates better management of an asthma treatment by the patient and their health care provider, and improves recognition of specific locations that precipitate rescue events so that the patient may avoid or accommodate these locations in the future.

### BRIEF DESCRIPTION OF DRAWINGS

FIG. 1 shows an asthma analytics system for monitoring accurate, real-time medicament device usage, performing analytics on that data, and providing asthma rescue event risk notifications, according to one embodiment.

FIG. 2 is a high-level block diagram illustrating an example of a computing device used in either as a client device, application server, and/or database server, according to one embodiment.

FIG. 3A shows a dashboard of a client application that allows a user to interact with an asthma analytics system, according to one embodiment.

FIGS. 3B shows an example card displayed within the dashboard of the client application, according to one embodiment.

FIG. 4 is an interaction diagram for providing asthma risk-based notifications based on medicament device monitoring, according to one embodiment.

FIG. 5 is a flowchart for detecting a rescue medication event by an asthma analytics system, according to one embodiment.

FIG. 6 is a block diagram illustrating the modules within the asthma risk module, according to one embodiment.

FIG. 7 illustrates a process for determining a patient's asthma risk using the components of the data analysis module 131, according to one embodiment.

FIG. 8 is a diagram illustrating a method for training a model using a training dataset, according to one embodiment.

FIG. 9A-B are diagrams illustrating visualization of an output generated by a non-linear model, according to one embodiment.

FIGS. 10A-B are diagrams characterizing and analyzing the data used for testing the asthma risk analysis, according to one embodiment.

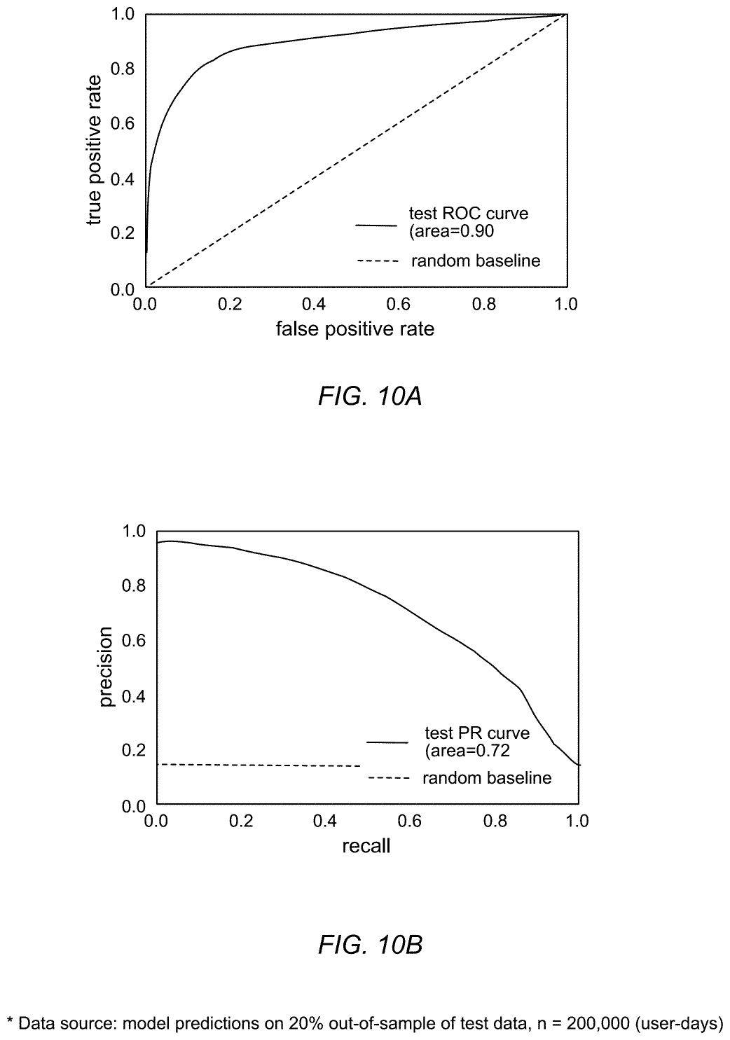

The figures depict various embodiments of the presented invention for purposes of illustration only. One skilled in the art will readily recognize from the following discussion that alternative embodiments of the structures and methods illustrated herein may be employed without departing from the principles described herein.

### DETAILED DESCRIPTION

### I. SYSTEM ENVIRONMENT

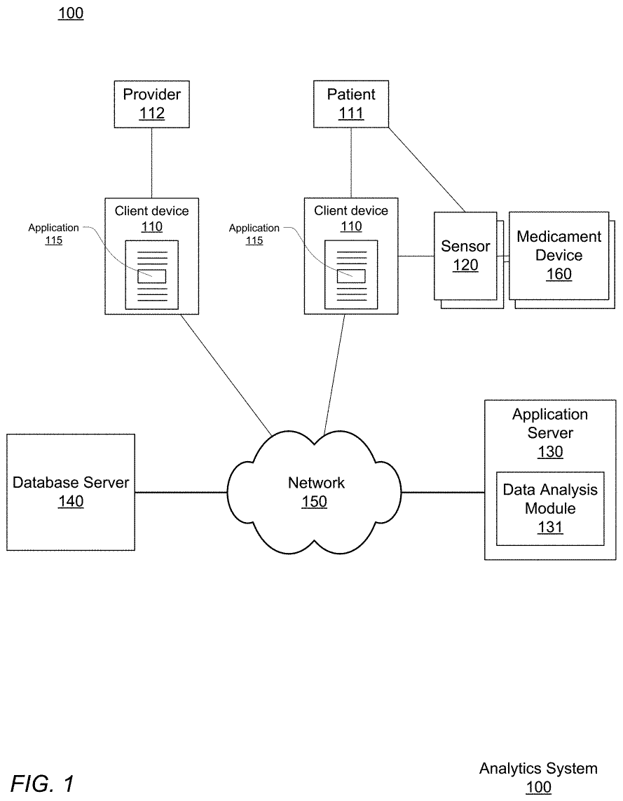

FIG. 1 shows an asthma analytics system 100 for monitoring accurate, real-time medicament device events, performing analytics on that data, and providing asthma rescue event risk notifications, according to one embodiment.

The asthma analytics system includes client computing devices 110, a medicament device sensor 120, a medicament device 160, an application server 130, database server 140, and a network 150. Although FIG. 1 illustrates only a single instance of most of the components of the asthma analytics system 100, in practice more than one of each component may be present, and additional or fewer components may be used.

I.A. Client Device and Application

The client devices 110, at the behest of their users, interact with the asthma analytics system 100 via the network 150. For purposes of explanation and clarity it is useful to identify at least two different types of users. A patient 111 is a user burdened with asthma who makes use of the asthma analytics system 100 at least in part to obtain personalized asthma rescue event risk notifications provided by the server 130 and asthma management notifications created by their health care provider 112. Such notifications can be provided in exchange for the user's permission to allow the asthma analytics system 100 to monitor the patient's 111 medicament device 160 usage. As will be explained below, medication events are detected by a sensor 120 associated with the medicament device 160 and the user's client device 100, which in turn reports to the application server 130, which in turn can initiate a process to generate risk notifications which are provided to the user through the client device 110.

Another type of user is a healthcare provider 112 who, again with the patient's 111 express permission, also receives notifications regarding a patient's asthma management, as well as aggregated asthma community rescue event data and derived statistics regarding asthma events and other associated data. Other types of users are also contemplated, such as parents/guardians of patients 111 who may also want to receive notifications in the event that their own client devices 110 are distinct from that of their children.

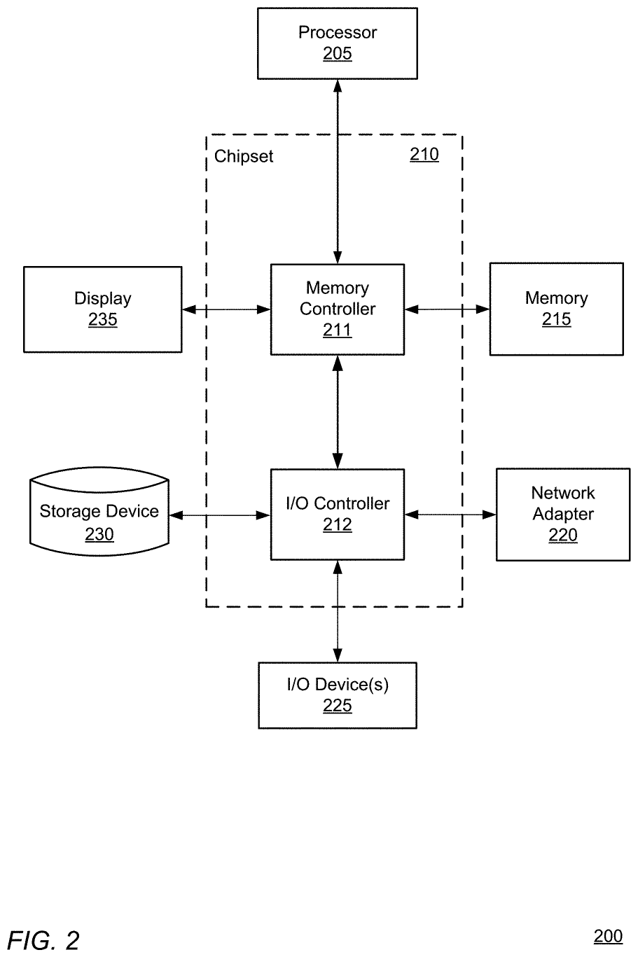

The client device 110 is a computer system. An example physical implementation is described more completely below with respect to FIG. 2. The client device 110 is configured to wirelessly communicate with the asthma analytics system 100 via network 150. With network 150 access, the client device 110 transmits to application server 130 the user's geographical location and the time of a rescue medication event, as well as information describing the event as received from the associated medicament device sensor 120 (referred to throughout as “sensor 120”).

Regarding user location and event times, the client device 110 may determine the geographical location and time of a rescue event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the sensor 120 as part of the event data or added to event data by querying an appropriate software routine available as part of the client device's native operating system.

In addition to communicating with the application server 130, client devices 110 connected wirelessly to the application server 130 may also exchange information with other connected client devices 110. For example, through a client software application 115, a healthcare provider 112 may receive a risk exacerbation notification describing a recent rescue event about a patient 111, then in response send a recommendation to the patient 111 for post-asthma rescue event treatment. Similarly, through application 115 patients 111 may communicate with their health care providers 112 and other patients 111.

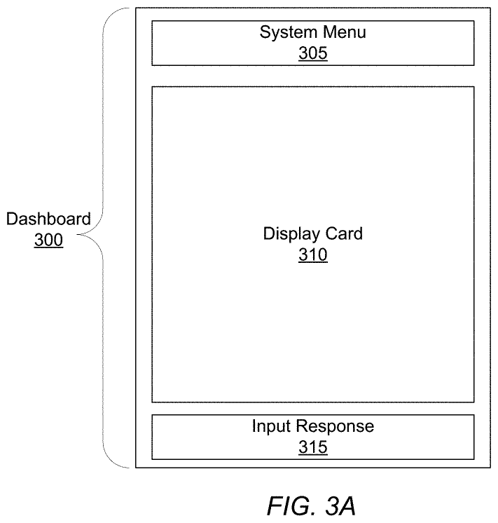

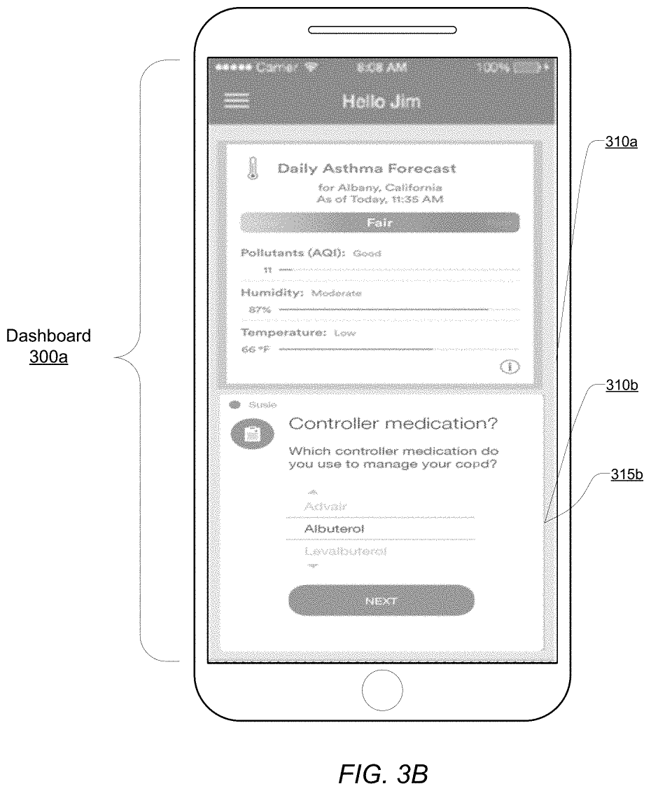

The application 115 provides a user interface (herein referred to as a “dashboard”) that is displayed on a screen of the client device 110 and allows a user to input commands to control the operation of the application 115. The dashboard is the mechanism by which healthcare providers 112 and patients 111 access the COPD analytics system 100. For example, the dashboard allows patients 111 and providers 112 to interact with each other, receive asthma rescue event risk notifications, exchange messages about treatment, provide and receive additional event and non-event data, and so on. The application 115 may be coded as a web page, series of web pages, or content otherwise coded to render within an internet browser. The application 115 may also be coded as a proprietary application configured to operate on the native operating system of the client device 110. The dashboard is more completely described below in conjunction with FIG. 3.

In addition to providing the dashboard, the application 115 may also perform some data processing on asthma rescue event data locally using the resources of client device 110 before sending the processed data through the network 150. Event data sent through the network 110 is received by the application server 130 where it is analyzed and processed for storage and retrieval in conjunction with database server 140. The application server 130 may direct retrieval and storage request to the database system 130 as required by the client application 115.

The client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicates information relating to medicament device usage with the client device 110. If the sensor 120 has not been paired with a client device 110 prior to a rescue medication event, the information is stored locally by the sensor 120 until such a pairing occurs. Upon pairing, the sensor 120 communicates any stored event records to the client device 110. In other implementations, other types of wireless connections are used (e.g., infrared or 802.11).

Although client devices 110 and medicament devices 160 are described above as being separate physical devices (such as smart phones and inhalers, respectively), the medicament devices 160 may include not only sensors 120 integrated into a single housing with the device 160, but also aspects of the client device 110. For example, a medicament device 160 may include an audiovisual interface including a display or other lighting elements as well as speakers for presenting visual audible information. In such an implementation, the medicament device 160 itself may present the contents of notifications provided by the server 130 directly, in place of or in addition to presenting them through the client devices 110.

I.B. Medicament Device and Sensor

The medicament device 160 is a medical device used to deliver medication to the lungs of a user experiencing constricted respiratory airflow. Medicament devices (e.g. inhalers) are typically portable and small enough to be carried by hand for ease of accessibility when treating respiratory attacks. In one embodiment, medicine is delivered in aerosol form through a medicament device 160 such as a metered dose inhaler. Metered dose inhalers include a pressured propellant canister of aerosol medicine, a metering valve for delivering a regulated medicine dosage amount, and a plastic holder that holds the pressurized canister and also forms a mouthpiece for delivery of the medicine. In another embodiment, medicine is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Examples of controller medications that are dispensed by a controller medicament device 160 include beclomethasone, budesonide, and fluticasone as well as combinations of those medications with a long-acting bronchodilator such as salmeterol or formoterol. Examples of rescue medications that are dispensed by a rescue medicament device 160 include albuterol, salbutamol, levalbuterol, metaproterenol, and terbutaline.

Each patient may be associated with more than one medicament device 160. For example, the patient may have a rescue medicament device 160 that dispenses rescue medication, and a controller medicament device 160 that dispenses controller medication. Similarly, each patient may be associated with more than one sensor 120, each chosen to operate with one of the patient's medicament devices 160.

Generally, a sensor 120 is a physical device that monitors the usage of the medicament dispenser 160. The sensor 120 is either removably attachable to the medicament dispenser without impeding the operation of the medication dispenser, or the sensor 120 is an integrated component that is a native part of the medicament dispenser 160 as made available by its manufacturer.

The sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter. However, in other embodiments, other types of wireless communication may be used (e.g., infrared, 802.11).

The sensor 120 may also be configured to communicate more directly with the application server 130. For example, if the network adapter of the sensor 120 is configured to communicate via a wireless standard such as 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130, for example using redundant transmission to ensure event data arrives at the application server 130 or to provide information directly to the client device 110 while the application server 130 is determining what notification to provide in response to an event.

As introduced above, the sensor 120 captures data about usage of the medicament device 160. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111 or health care provider 112. The medication event information is sent to the application server 130 for use in analysis, generation of asthma rescue event notifications, and in aggregate analyses of event data across multiple patients.

To accomplish this goal, there are a number of different ways for the sensor 120 to be constructed, and in part the construction will depend upon the construction of the medicament device itself 160. Generally, all sensors 120 will include an onboard processor, persistent memory, and the network adapter mentioned above that together function to record, store, and report medication event information to the client device 110 and/or server 130. Sensors 120 may also include a clock for recording the time and date of events.

Regarding specific sensor 120 constructions, traditional inhalers, such as mechanical dose counters, are not designed with sensors 120 in mind, and thus the sensor 120 may be constructed accordingly. Some implementations in this manner include mechanical, electrical, or optical sensors to detect movement of the device 160, priming of the device, activation of the device, inhalation by the user, etc. In contrast, modern inhalers, such as deflectable membrane dose counters, include electrical circuitry that may report event information as an electrical data signal which a sensor 120 is designed to receive and interpret. For example, the medicament device 160 itself may report movement, priming, and activation to the sensor 120. In one embodiment, the sensor detects movement of the medicament device, for example an opening in the medicament cover which indicates that medication is being dispensed. In alternate embodiments, the sensor may detect movement of the canister to a position from which medication is dispensed. After detecting such movements which indicate that the medicament device has been activated, the sensor may report that the medicament device has dispensed medication and the time at which it dispensed the medication.

More information regarding hardware and software components for the sensors 120 and medicament devices 160, as well as the interaction between them to record rescue medication events can be found in U.S. patent application Ser. No. 12/348,424, filed Jan. 1, 2009, and International Application No. PCT/US2014/039014, filed May 21, 2014, both of which are incorporated by reference herein in their entirety.

I.C. Application Server

The application server 130 is a computer or network of computers. Although a simplified example is illustrated in FIG. 2, typically the application server 130 will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use asthma analytics system 100 by many different client devices 110 through network 150, and thus at a high level can be generally characterized as a cloud-based system. The application server 130 generally provides a platform for patients 111 and healthcare providers 112 to report data recorded by the sensors associated with their medicament devices 160 including both rescue medication and controller medication events, collaborate on asthma treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server140 has been updated.

The application server 130 stores and manages data at least in part on a patient by patient basis. Towards this end, the application server 130 creates a patient profile for each user. The patient profile is a set of data that characterizes a patient 111 of the asthma analytics system 100. The patient profile may include identify information about the patient such as age, gender, current rescue medication, current controller medication, notification preferences, a controller medication adherence plan, a patients relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 and their personal healthcare providers 112, as well as the frequency with which notifications are provided. For example, a patient 111 may authorize a healthcare provider 112 to receive notifications indicating a rescue event. The patient 111 may also authorize their healthcare provider 112 to be given access to their patient profile and rescue event history. If the healthcare provider 112 is provided access to the patient profile of the patient 111, the healthcare provider may specify controller adherence or rescue medication plans. Medication plans may include a prescribed number of doses per day for controller medications.

The application server 130 also creates profiles for health care providers 112. A health care provider profile may include identifying information about the health care provider 112, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, yearly).

The application server 130 receives rescue medication event information from the client device 110 or the sensor 120, triggering a variety of routines on the application server 130. In the example implementations described below, the data analysis module 131 executes routines to access asthma event data as well as other data including a patient's profile, analyze the data, and output the results of its analysis to both patients 111 and providers 112. This process is generally referred to as an asthma risk analysis. The asthma risk analysis may be performed at any point in time, in response to a rescue event, due to a relevant change in the patient's environment, and in response to any one of a number of triggering conditions discussed further below.

Other analyses are also possible. For example, a risk analysis may be performed on rescue and controller medication use for multiple patients to identify based on spatial/temporal clusters (or outbreaks) of medication use based on historically significant permutations from individual, geographic, clinical, epidemiologic, demographic, or spatial or temporal baselines or predicted or expected values. Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations.

Responsive to any analyses performed, the application server 130 prepares and delivers push notifications to send to patients 111, authorized healthcare providers 112, and/or other users provided access to the patient's profile. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the asthma risk analysis performed by the data analysis module 131. More information regarding the types of notifications that may be sent and the content they may contain is further described below.

In addition to providing push notifications in response to an asthma risk analysis, notifications may also be provided as pull notifications, at particular time intervals. Additionally, some notifications (whether push or pull) may be triggered not in response to an asthma risk analysis performed in response to a rescue medication event, but instead in response to a risk analysis performed in response to one of the underlying factors in the asthma risk analysis changing, such that an updated notification is warranted. For example, if weather conditions indicate that an increase in air pollution is occurring or is imminent, this may trigger the carrying out of asthma risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

I.D. Database Server

The database server 140 stores patient and provider data related data such as profiles, medication events, patient medical history (e.g., electronic medical records). Patient and provider data is encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy.

The database server 140 also stores non-patient data used in asthma risk analyses. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130 the database server 140 may alternatively be a hardware component that is part of another server such as server 130, such that the database server 140 is implemented as one or more persistent storage devices, with the software application layer for interfacing with the stored data in the database is a part of that other server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with users, groups of users, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

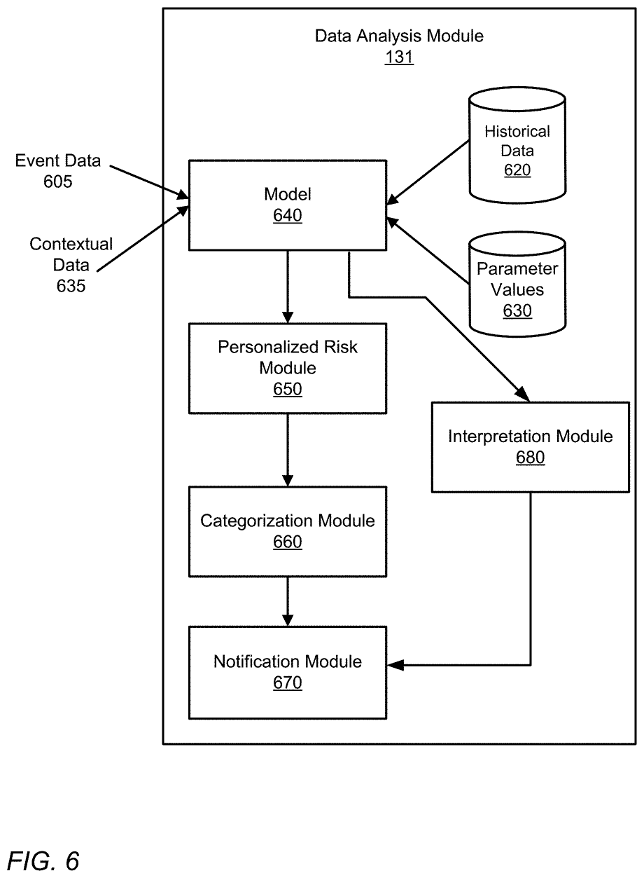

With respect to the description of FIGS. 6A-6D below, the contents of the databases described with respect to those figures may be stored in databases physically proximate to the application server 130 and separate from database server 140 as illustrated. Alternatively, those databases may be a part of database server 140, in contrast to the description of FIGS. 6A-6D illustrating them as being within data analysis module 131. This and other variations thereupon are within the scope of this description.

I.E. Network

The network 150 represents the various wired and wireless communication pathways between the client 110 devices, the sensor 120, the application server 130, and the database server 140. Network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

### II. EXAMPLE COMPUTING DEVICES

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard driveor a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)).

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

### III. DASHBOARD

The dashboard, for example dashboard 300 illustrated in FIG. 3A, allows users to interact with the asthma analytics system 100. The dashboard 300 provides a means to transfer information on a user-to-user (e.g., patient 111 to provider 112) or user-to-system/system-to-user basis. Dashboards 300 are accessed through the client application 115 on the client device 110 and provide a mechanism for both patients and healthcare providers to monitor medication rescue events, exchange personalized patient healthcare information, and receive notifications such as asthma rescue event risk notifications. Patients may communicate with other health care providers and other patients through the dashboard 300, for example, to discuss and share information about asthma, medication usage, or asthma management. The ability to share asthma healthcare information may give patients or healthcare care providers experiencing a similar issue a way to share individual perspectives.

The dashboard 300 also allows authorized health care providers 112 to access a list of patients to view, annotate, update, interact with, and export information about asthma patient and community data and statistics in various demographics or geographic segments. Using the dashboard 300, healthcare providers are able to monitor patients individually or in aggregate, to receive and provide feedback on how their associated patient populations are responding to asthma management guidance. A healthcare provider who has access to individual or multiple patients has the ability to establish notification thresholds, set parameters for the notifications, and receive notifications when patients' event history matches certain conditions (e.g. rescue event). Additionally, the dashboard 300 can receive and display regular reports of event patterns for specific demographic generated by the asthma analytics system 100.

The dashboard 300 presents a variety of information including tabular data, graphical visualizations, and analyses to users through display “cards” 310. Display cards 310 are conformably suited to smaller displays typical of portable client devices 110, for example mobile phones or tablets, and include “bite size” pieces of information that mimic the simplistic organizational style found in baseball cards. The dashboard 300 may also include a system menu 305 that allows users to navigate through different categories of healthcare information.

Notifications provided by the application server 130 are related to the display cards 310. Generally, notifications include not only information to be presented to the user through the application 115, but also parameters for specifying which display card 310 is to be used to display the contents of the notification. Any information pushed/pulled from the application server 130 may be associated with one or more cards. For example, a notification can be pushed to the patient based on the outcome of an asthma risk analysis. The dashboard 300 will process the notification and determine which card/s to use to present the information in the notification. Continuing the example, the recipient of the notification may make a request to pull data from the application server 130. The application server 130 provides the requested data in another notification, and the dashboard 300 then determines which display card 310 to display the requested information.

To interact with information presented, some display cards 310a include an input response 315 area. For example, in the display card 310b illustrated in FIG. 3B, a patient may scroll up or down in the input response 315 area to select a controller medication used to manage asthma or select the “next” to move to an additional display card 310.

The dashboard 300 may provide a variety of different display cards 310, which may be organized into categories. An information card type includes cards that display data. Information cards may, for example, display medication rescue events, statistics, and maps including both patient data and community data. Information cards are further sub-categorized into event, trend, education, and alert display cards.

Event cards include data relating to rescue medication events, such as a list of historical medication rescue events for a specific patient, or patient rescue event data overlaid on a geographical map for a specific provider.

FIG. 3B illustrates an example display card 310a sent by the notification module 645 (discussed below), which is sent based on the determination of a risk score by the data analysis module 131. The display card 310a highlights patients information obtained from the input values used to determine the risk score, for example the patient's current geographical location as specified by their device 110, and environmental information. The display card 310a also includes a risk categorization based on the risk score, in this case “fair” which may represent a medium risk categorization.

Another event card may display an example medication usage report including a map of the location of a rescue usage event, environmental conditions at the location, and an input response area 315 for the recipient to add triggers for the rescue usage event. Another event trend card may display rescue device usage for the previous week including a total number of uses for the time period and a number of uses for each day.

A trend card includes statistical information presented using a graph or a chart designed for clear comprehension by the recipient. Examples of trend cards include plots of asthma rescue events over various time periods, time of day trends, days of week trends, and trigger trends.

An education card includes information meant to educate the recipient. Education cards provide general disease information and tips for patients to reduce their risk of rescue events. Some education cards may require an input response 315 to allow recipients to specify whether the information provided is relevant or interesting for use in serving future cards.

Alert cards notify users of important information including informing a recipient that they are at risk for an event and/or that data has not been received from a device over a recent time period. Other alerts may include an alert that a setting on the client device is preventing data syncing (e.g. Bluetooth is turned off) or that a patient's asthma risk score has changed.

A survey card type solicits a user response by presenting yes/no, multiple choice, or open-ended questions for the user. For example, a healthcare provider or the asthma analytics system 100 may send a survey card with an asthma-related questionnaire to a patient 111 to determine a level of disease control for a specific patient. Additionally, a survey card may request the type of controller medication that the patient 111 uses to treat asthma symptoms. Generally, survey cards provide the application server 130 with data that may be missing from a patient's medical history or patient profile (as introduced above), and/or provide an update to potentially outdated information. One or more survey cards may be used to complete the patient enrollment process and create a patient profile for storage in database server 140. For example, when a patient 111 initially enrolls in the asthma analytics system 100, a push notification will be triggered by the application server 130 prompting the patient 111 to create a patient profile.

Example of survey cards may include a survey question asking whether a patient has made any emergency room visits as a result of asthma rescue events, information about the patient's controller medication, how many times the patient used their rescue medication to control an event, and what their controller medication daily schedule is. Survey cards may also ask about a patients asthma triggers, such as whether pollen is a trigger. Some survey cards may ask a patient to rate their general quality of life on 5-point Likert scale, to rate their quality of sleep, to rate their ability to be active over last 7 days, and so on. Other survey cards ask whether the patient feels better or worse than yesterday, whether the patient has had to go to emergency room or hospital in last 12 months for a rescue event and so on.

In some instances, patient behavior or sensor-reported event information that is inconsistent with existing patient information may trigger the sending of a survey card in order to resolve ambiguity as to the patient's circumstances. For example, if the patient is experiencing a greater-than-expected count of asthma events, the survey card may request information about the type of medication the patient has currently listed on their medicament device 160, in order to verify that the correct medication is being used. Another example includes if the reported information about controller medication use indicates that the patient is only using the controller medication one time per day but their adherence plan indicates they are supposed to be taking their controller medication twice per day, system 100 could send a notification asking if the patient needs to change their adherence plan.

In some instances, patient behavior or sensor-reported event information that is inconsistent with existing patient information may trigger the sending of a survey card in order to resolve ambiguity as to the patient's circumstances. For example, if the patient is experiencing a greater-than-expected count of asthma events, the survey card may request information about the type of medication the patient has currently listed on their medicament device 160, in order to verify that the correct medication is being used. As another example, if the reported information about controller medication use indicates that the patient is only using the controller medication one time per day but their adherence plan indicates they are supposed to be taking their controller medication twice per day, system 100 could send a notification asking if the patient needs to change their adherence plan.

Navigation cards represent actionable data or messages that, upon user interaction, may redirect the user to another screen or card that is part of the dashboard 300. For example, if a patient wants to share information or request specific medication plans for controller medications with a physician, a navigation card would be used to facilitate the sharing of information or enrollment in controller adherence plan. Additionally, navigation cards allow users to update information surrounding medication rescue events.

Adherence cards are designed to encourage a patient to continually use their controller medication on schedule over different periods of time. Adherence cards may indicate a “streak” or continuous run of on-time controller medication events. Additionally, a survey card may inquire as to the patient physical state in response to recording a significant number of rescue events within a threshold period of time of each other. Controller medication events may be represented as graphs to illustrate when the patient 111 did and did not take their controller medication on time during various periods during the day, as prescribed by their health care provider 112. Cards may also detail a daily schedule for controller medication and an indicator for displaying whether the scheduled dose has been taken. For example, a red “X” may indicate the scheduled dose has not been taken, but a green check mark or a different symbol may indicate that the scheduled dose has been taken.

Setup cards guide recipients in associating sensors with client devices 110. Setup cards may guide pairing a sensor to a client device 110 using Bluetooth, prompt the recipient to initiate the pairing process or prompt the user to select a sensor device for pairing, or notify the user that the sensor is paired.

In some embodiments, the dashboard may present a user interface. The user interface may illustrate a list of rescue events, responsive to the user's selection of the “View Timeline” input response area 315c. The list displays rescue usage events over a time period and includes details such as date, time, number of puffs, and location. Recipients may edit rescue usage events and/or add additional details by selecting the edit interaction response areas. Some interfaces may present an event summary for a rescue usage event to the user. The event summary may be presented to the user in response to the user selecting the edit interaction response area of the user interface. From the dashboard, the user may also view and edit a medication list, detailing information such as medication type (rescue, controller, etc.), dosage schedule, and sensors.

### IV. EVENT-DRIVEN ASTHMA RISK NOTIFICATIONS

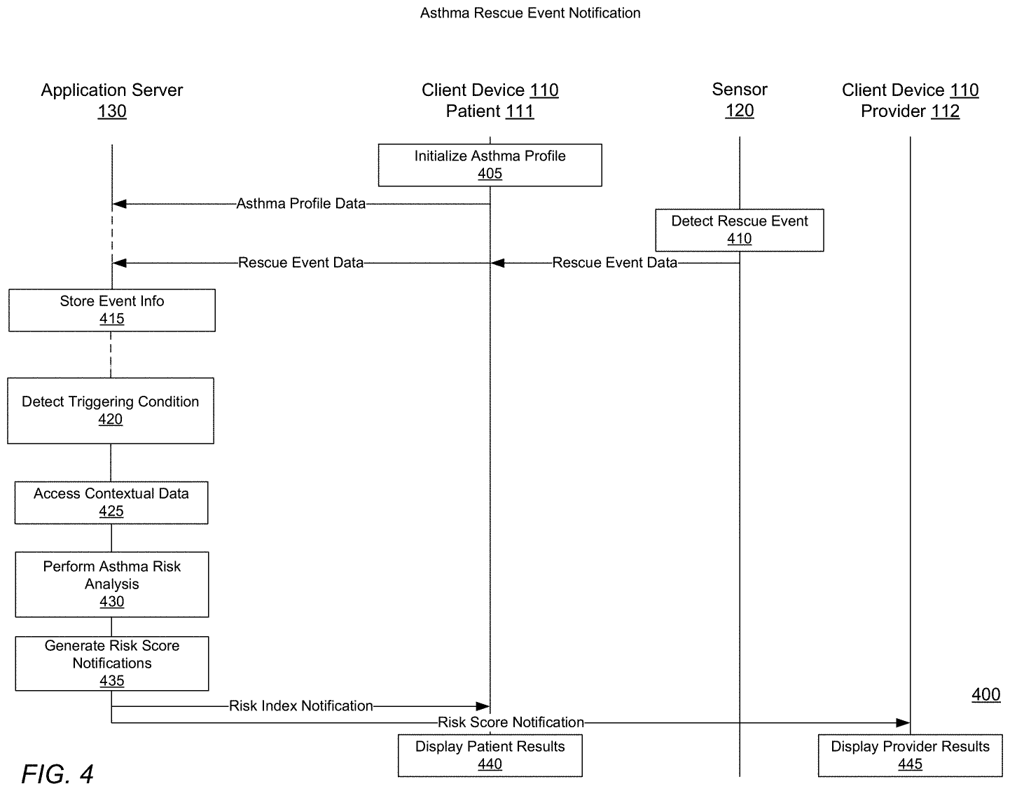

FIG. 4 shows an interaction diagram for providing asthma risk notifications based on medicament device monitoring, according to one embodiment. As an initial step, a patient interfaces with the dashboard 300 to initialize 405 a patient profile. Once the patient completes their patient profile, the client device 110 transmits the patient profile for use by the application server 130 and storage by the database server 140. Once a patient's patient profile is initialized 405, the application server 130 may begin to receive rescue medications events detected by the sensor 120 associated with the patient's medicament device 160. The initializing and completing of the patient profile is only performed once during the patient's first use of the medicament device.

Upon the sensor detecting 410 a rescue usage event, the client device 110 collects and sends the rescue event data to the application server 130, where the event information is stored 415. Although only one such instance is shown in FIG. 4, this detection and storage process is generally performed with some frequency for many patients, generally upon detection of a rescue usage event. However, this frequency may differ from the frequency with which a risk analysis is performed.

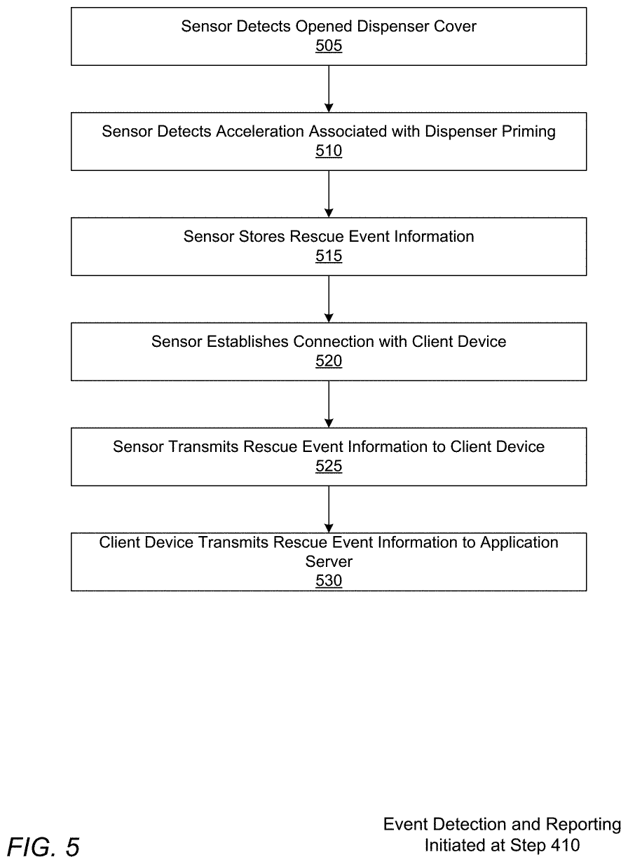

Referring now to FIG. 5, the application server 130 generally receives a rescue event anytime the patient uses their rescue medicament dispenser 160 to relieve asthma-related event symptoms. As an example of the process for capturing such an event for a particular device 160/sensor 120 combination, at the start of symptoms, the sensor 120 may detect 505 whether a medication dispenser 160 cover is opened. When the medication dispenser cover is opened, the sensor 120 may detect an acceleration 510 associated with a priming of the dispenser 160. For some types of medicament dispensers, the “priming” includes activating a mechanism to release a dose of a medication from a packaging. In other types of medicament dispensers, the “priming” includes a rapid shaking of a medication canister.

After the priming action is detected, the sensor 120 is configured to store 515 data associated with the rescue event in active memory of the sensor 120. The rescue event data may include information that describes the time and date of associated with the rescue event, the status or condition of the medicament device 160 (e.g. battery level), the number of doses of medication remaining (before or after the event), self-test results, and physiological data of a patient being treated with the medicament device 160 as measured by the sensor 120. When the sensor establishes a network connection with either the client device 110 or network 150, the sensor transmits 525 any locally stored rescue event data to the client device 110 or the application server 130. If the event data was transmitted to the client device 110 first, the client device 110 then transmits 530 the rescue event data to the application server 130 when the client device 110 establishes a network connection with the network 150. Depending upon the implementation, either the client device 110 or sensor 120 will add the geographic location where the event took place to the event data transmitted to the application server 130.

Returning now to FIG. 4, upon detecting 410 a rescue usage event, event data is collected and stored 415. Upon receiving and storing 415 the rescue usage event data, the application server 130 may request further information from the patient describing the rescue usage event. To obtain the information, the application server 130 generates a push notification, including the questions to be asked, to be sent to the patient's client device 110. The client device 110 may present the push notification as a survey type card 310. The patient may respond to the request by providing inputs 315 in response to the survey card 310. Alternatively, the patient 111 may elect not to respond to the request. This is permissible and any gaps in information may be obtained through later push notifications, or upon entry by provider 112 after a meeting with the patient 111. In one implementation, the failure to receive the additionally requested data in response to request does not hold up the remainder of the analysis described in steps 425-445.

Based on the information collected as part of the event or otherwise, the asthma analytics system detects 420 a triggering condition. Triggering conditions refer to information pertaining to parameters that may have played a role in triggering the event, a location of the rescue event, a label (e.g., work, home, or school) for the location, a rating to signify the personal importance of the location to the patient, and whether the use was pre-emptive (e.g., medication taken prior to exercising) in addition to any other relevant information. Examples of triggering conditions include, but are not limited to, a change in a location of the client device, a conclusion of a period of time for recording rescue inhaler usage events, change in a value of at least one patient parameter, weather parameter, and air pollutant parameter, and an occurrence/detection of a rescue inhaler unit dispensing rescue medication.

In addition to requesting additional event data, the application server 130 accesses 425 stored contextual data from the database server 140. Generally, contextual data refers data other than event data, which includes but is not limited to: to atmospheric conditions, weather conditions, patient data recorded from past rescue usage events, and any other considerations that are not directly detected by the medicament device at the time of the rescue usage event. By contrast, event data refers to any parameters related to the rescue event and reported by the medicament device, such as medication dosage, the time of the event, the location of the event, and relevant adherence data. Both forms of data may include temporally-current information as well as stored historical data. Accordingly, as part of obtaining the contextual data, the application server 130 also accesses historical rescue usage event data for the patient 111. This historical data can include all of the data from any past controller or rescue medication event data from the patient's history over a variety of windows of time in the past, and each historical event may include all of the same items of information as was reported 410 for this event along with any data collected upon follow up thereafter.

However, note that in the following description, such as in FIG. 6, in some instances contextual data 635 and historical data 620 are represented separately. The contextual data 635 refers to geographic and regional information relevant to the current event or current location of the patient's client device 110, whereas historical data 620 refers to geographic, regional, and event information from previous rescue usage events from the same or different patients.

IV.A. Asthma Prediction Model Overview

FIG. 6 is a block diagram illustrating the logical components that carry out the functions of the data analysis module 131, according to one embodiment. The asthma analytics system 100 includes, on the application server 130, a data analysis module 131 that analyses the variety of data collected by the system, introduced above and discussed further below, to perform 430 risk analyses for patients upon occurrence of a triggering condition. These risk analyses are used to generate notifications that are specifically configured to be sent to a patient in a sufficiently timely manner to reduce occurrences of an asthma event that would necessitate usage of their rescue inhaler. In the illustrated embodiment of FIG. 6, the data analysis module 131 includes a historical data store 620, a parameter value store 630, a model 640, a personalized risk module 650, a categorization module 660, a notification module 670, and an interpretation module 680. In other embodiments, the data analysis module 131 may include more or fewer modules. Functionality indicated as being performed by a particular module may be performed by other modules or components instead.

A risk analysis may be triggered by a triggering condition, which itself may be automatically scheduled, manually set, and/or occur responsive to particular event or contextual information. Examples of triggering conditions include but are not limited to: a change in an input value, a change in the location of a user, a result of the occurrence of a rescue event, or a conclusion of a time interval.

In order to perform a risk analysis, a model 640, such as a mathematical function or other more complex logical structure, is trained using historical event data and historical contextual data to determine a set of parameter values that are stored in advance and used as part of the risk analysis. Parameter values 630 describe the “weight” (or critical value or other similar quantity, depending upon the modeling technique used) that is associated with at least one of the input values. Input values refer to numerical or categorical values of the parameters of the model 640, where the input values vary between patients over time.

Briefly, the risk analysis is performed by inputting aspects of a user's rescue inhaler usage events 605, 620 and the other contextual data 635, 620 as input values to the model's 640 function and parameter values 630 and determine a numerical risk score. For example, the model 640 receives, as inputs, a combination of weather data, pollutant data, patient data, social data, and built environmental data, and generates, as an output, a prediction of a number of medication puffs taken by a patient. Generally, the contextual data (i.e., weather data, pollutant data, patient data, social data, and built environmental data) collected for use as the input values may be gathered automatically based on device or other third party data reporting, manually provided by a patient and/or provider, or otherwise obtained.

The expected number of puffs (e.g., an amount of mediation dispensed by a rescue inhaler unit) is a numerical value, generated by the model 640, that characterizes the number of expected medication usage rescue events that a patient will experience for the current day given event data 605 and contextual data 635. A record of a patient taking a mediation puff is representative of a patient experiencing a rescue medication usage event. Since a prediction of a large number of puffs correlates with an increased number of expected rescue usage events, a prediction of the number of puffs may be interpreted as representative of an asthma risk assessment for a patient on a given day. In an alternative embodiment, the model may instead generate a probability that a patient will experience an expected number of puffs (e.g., a predicted number of rescue inhaler usage events) above a threshold determined for an entire population of patients. The population of patients may be identified based on proximity or similarities in their medication usage.

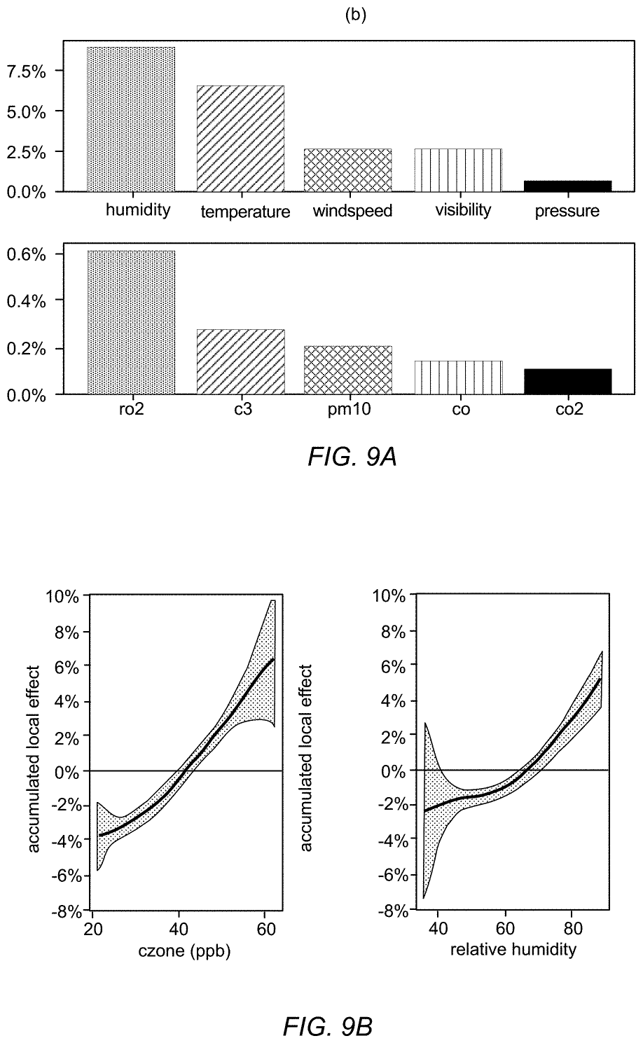

In some implementations, information from the trained model 640 and/or specific inputs and predictions for a user are communicated to an interpretation module 680. The interpretation module 680 performs various processing techniques to further visualize the internal operations of the model 640. These techniques include, but are not limited to, accumulated local effects and permutation-based feature importance at the population level, or Shapley additive explanations at the sample level or individual patient level. Accordingly, the interpretation module 680 may provide additional insights to a patient or provider as content in a notification generated by the notification module 670, providing visual explanations of how individual environmental variables may influence rescue inhaler usage across the entire patient population, for an individual patient, or for the individual patient's current day. FIG. 9A illustrates an example of a feature importance visualization, FIG. 9B illustrates an example of an individual variable effect visualization, according to some embodiments.

The prediction generated by the model is received by a personalized risk module 650 to compute a patient-specific risk score. In some embodiments, the patient-specific risk score is determined based on a comparison of the raw prediction generated by the model 640 to a recent historical baseline generated for that patient. The historical baseline may be computed using a variety of techniques to determine a representative distribution of puffs over a period of time. The period of time upon which the historical baseline is computed may be a set of consecutive days preceding a current day for which the model 640 predicted an expected number of puffs. In another embodiment, instead of the daily predicted number of puffs, the historical baseline for a patient may be computed based on a patient's daily recorded medication usage over a preceding period of time.

In one embodiment, the personalized risk module 650 compares the output of the model for the current day (i.e., the predicted number of puffs for the current day) as a percentile of historical predictions of the number of puffs for a period of preceding days. In practice, although each historical prediction may carry a degree of error, because that error is roughly symmetric in nature, the current prediction may be compared to the entire distribution of historical predictions to accurately characterize a patient's risk score.

In an alternate embodiment, the personalized risk module 650 determines the historical baseline by computing a standard deviation of actual historical puffs for each day over a preceding period of time. For example, the personalized risk module 650 calculates a z-score by subtracting the mean puffs of the past 30 days and dividing the difference by the standard deviation. The personalized risk module 650 may apply an additional computational technique, for example a sigmoid function, to characterize the patient-specific risk score (i.e., the comparison between the historical baseline and the predicted number of puffs) as a value between 0 and 1.

In another alternate embodiment, the personalized risk module 650 determines the historical baseline using bootstrap methods. The module first calculates a distribution of sample means based on repeated random samples with replacement from the baseline period of historical puffs per day, and then calculates the expected number of puffs as a percentile of this distribution.

The patient-specific risk score generated by the personalized risk module 650 is received by the categorization module 660. The categorization module 660 categorizes the patient-specific risk score into a range of risk scores, for example, “good”, “fair,” or, “poor.” In some embodiments, the model 640 characterizes the comparison between two days as a fraction comparing a number of doses or a percentile comparing a history of risk scores. For example, a “good” categorization may apply to patient-specific risk scores ranging from 0-0.3, a “fair” categorization may apply to patient-specific risk scores ranging from 0.3-0.9, and a “poor” categorization may apply to patient-specific risk scores ranging from 0.9+.

In particular implementations, the model 640 receives air pollutant data indicating that the air quality for a particular geographic region poses a significant risk to patients at risk of asthma related events. Such instances of air pollutant data may be identified using an index or look-up table provided by a third-party air quality analysis provider or the National Oceanic and Atmospheric Administration (NOAA). In such instances, the model 640 implements a combination of rule-based processing techniques to generate and communicate a notification with a “poor” categorization to all at-risk patients within the affected geographic region.

In another implementation, the categorization module 660 compares a patient's actual puff count for a given day to a threshold cumulative puff count generated by the personalized risk module 650. The actual puff count may be recorded based on periodic signals received from a sensor coupled to a patient's rescue usage inhaler unit that indicate the inhaler unit was used to administer a puff. If the actual puff count meets or exceeds the threshold, the categorization module 660 categorizes the patient specific risk score as “poor” regardless of the value of the actual numerical value of the risk score for the remainder of the day. Such a threshold may be determined based on historical data of rescue usage events recorded for a patient over a preceding period of time. For example, at a given time on a given day for a given user, the expected number of daily puffs may be 1.2, which may translate to a patient-specific risk score of 0.2. Although, a day with a risk score of 0.2 may normally be categorized as “good,” because the actual puff count of 4 exceeds a threshold of 3 puffs, the day is instead categorized as “poor.”

IV.B. Asthma Risk Analysis Implementation

FIG. 6B illustrates a process for determining a patient's asthma risk using the components of the data analysis module 131, according to one embodiment. A model stored at the data analysis module 131, for example the model 640, receives 710, as inputs, a combination of weather data, pollutant data, patient data, social data, and built environmental data. The model is trained to generate, as an output, a prediction of a number of medication puffs taken by a patient for a day corresponding to the received contextual parameters (i.e., the weather data, pollutant data, patient data, social data, and built environmental data). Using the model 640, the data analysis module 131 generates 720 a predicted number of puffs that the patient will take over the given day. Accordingly, a higher predicted number of puffs may indicate that a patient is more susceptible to rescue asthma usage events. The output of the model may also be a binary value (i.e., “0” or “1”) representing whether the predicted number of puffs exceeded a population-based threshold, for example a threshold determined based on data for population of users included in the dataset on which the model was trained.

The data analysis module 131 determines 730 a historical baseline based on a patient's asthma-related history. For example, the module 131 may determine the historical baseline based on puff predictions generated by a model for a set of preceding days, for example the previous week's worth of predictions. Alternatively, the module 131 may determine the historical baseline based on a patient's actual medication usage over a preceding period of time (i.e., how often the patient used their medicament device).

The data analysis module 131 determines 740 a patient-specific risk score by comparing the predicted number of puffs for the current day to the historical baseline. In some embodiments, the patient-specific risk score is a numerical value ranging between 0 and 1. Although the patient-specific risk score is a numerical value, it may also be characterized into a risk category. The data analysis module 131 may further compare the patient-specific risk score to a set of thresholded ranges between 0 and 1 to categorize 750 the patient-specific risk score as either “good,” “fair,” or “poor.” The categorization is communicated 760 by the data analysis module 131 to a patient as a notification received via their client device. In addition to the categorization of their asthma risk score, the notification also includes a recommendation outlining options for a patient to improve their asthma risk.

IV.C. Model Training

With regard to model training and FIG. 6A and 6B, the model is trained on a training dataset of prior days labeled based historical data, where each prior day is associated with a label based on historical event data and historical contextual data recorded during the prior day. In one embodiment, the label assigned to a prior day in the training dataset represents an indication of the actual number of puffs recorded for a patient on the prior. As described above, during the training of the model 640 weights are determined for each contextual parameter value that describe the relative significance of each parameter in determining a puff prediction for a given day. In one embodiment, the weights assigned to parameters are numerical values ranging between 0 and 1, where weights closer to 0 are assigned to less significant contextual parameters and weights closer to 1 are assigned to more significant contextual parameters. During the implementation of the model 640 for a given test day, the model 640 (including the weights assigned to each parameter during training) are accessed. For each contextual parameter, the data analysis module 131 accesses a measurement characterizing the contextual parameter on the given data. Each accessed measurement, also referred to as an input value, is input to the trained model 640.

To create the training dataset used to train the model 640, the data analysis module 131 aggregates or segments accessed historical data into periods of time preceding a current day (also referred to as “trailing windows”), and identifies input values for the various contextual parameters of the model 640. In addition to the preceding set of days, the trailing window may also include environmental parameters for the current day. For example, if a patient-specific risk score is computed for Day 8 at 10:00 AM, the trailing window may include environmental data, controller usage information, and rescue usage information associated with Days 1-7 and environmental data recorded cumulatively throughout Day 8 between 12:00 AM and 9:59 AM. In an alternate implementation, the trailing window may include environmental data, controller usage information, and rescue usage information associated with Days 1-7 and current environmental information recorded on Day 8 at 10:00 AM. Environmental information, for example air quality and weather data may be obtained from National Oceanic and Atmospheric Administration (NOAA) and Environmental Protection Agency (EPA) historical datasets.

In another embodiment, each day in the training dataset is assigned a binary label (e.g., a “1” or “0”), indicating whether or not the number of puffs recorded for that day is above or below a threshold value. In such embodiments, the threshold is a fixed number of puffs that is set for the entire population of patients, rather than a particular patient. As described above, in one exemplary implementation, the threshold is determined as an average of the number of puffs recorded for each patient of the training dataset. Accordingly, a model which receives inputs with a binary label outputs a probability that a patient will experience a number of rescue puffs above a population-based threshold number of rescue puffs.

The population-based threshold may be determined based on the total number of puffs recorded for a population of patients over a specified prior time period preceding either the current day for which the predicted number of puffs is being determined, or during a time period preceding the time of a current or most recent rescue usage event. In one implementation, the threshold is a fraction (percentage) of the total number of usage events over a specified time period. However, in other embodiments, other functions of the total number of usage events may be used to determine the threshold.

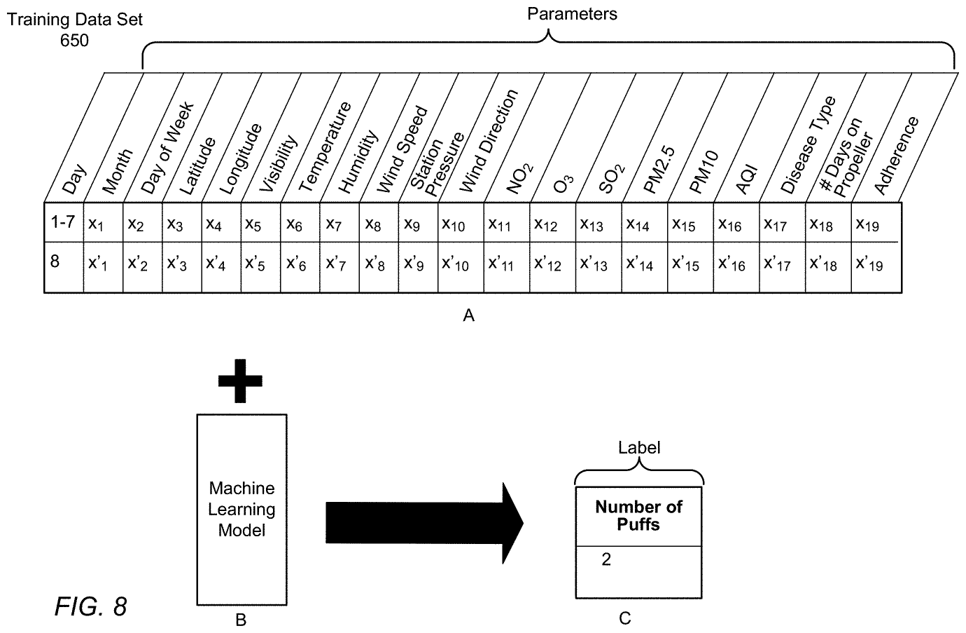

FIG. 8 is a diagram illustrating a method for training the model 640 using a training dataset, according to one embodiment. The model 640 is trained by determining parameter values 630 (e.g., associated with each parameter, not shown) that best represent the relationship between the input values (cells of the table A) of the training samples 650 (each row of the table) and the labels of those samples (C). As introduced above, the model 640 is trained using some function (B) or another more complex logical structure. In one embodiment, the model 640 is trained using a machine learning technique, examples of which include but are not limited to linear, logistic, and other forms of regression (e.g., elastic net), decision trees (e.g., random forest, gradient boosting), support vector machines, classifiers (e.g. Naïve Bayes classifier), fuzzy matching, and neural network sequence models (e.g., long-short term memory recurrent neural networks and time convolutional networks). An example model 640 trained using a long-short term memory network is described below.

Once the parameter values are known, the model 640 may be used by accessing the parameter values and the function specified by the model, and inputting input values for the parameters to generate an expected number of puffs for a patient on a given day. In embodiments in which the model is trained on a training dataset comprised of days assigned binary labels, the model 640 may be used by accessing the parameter values and the function specified by the model, and inputting input values for the parameters to generate a probability that a patient will experience an above-threshold number of puffs on a given day.

In the illustrated embodiment of FIG. 8, the training dataset (A) includes data describing environmental and patient parameters for seven immediately preceding days and environmental parameters for an eighth, current day. Each parameter for each of the eight days is an individual input to the machine-learned model (B). Accordingly, for each day during the training set the model (B) outputs a numerical value which is assigned label (C). The label (C) assigned to the model's output for a given day may be a real label specifying the predicted number of puffs (i.e., 2 puffs as illustrated in FIG. 8), or a binary label specifying the probability that the predicted number of puffs is above or below a threshold number of uses. Additionally, it would be obvious to one having skill in the art that the parameters illustrated in FIG. 8 are meant to be exemplary without limiting the scope of potential parameters.

IV.D. Input Parameters

The parameters incorporated into the risk score analysis can be categorized into several groups: historical patient parameters, current patient parameters, air pollutant parameters,weather parameters, and contextual parameters. Historical and current patient parameters may be more broadly categorized as simply “patient parameters”. Air pollutants and weather variables may be more broadly categorized as simply “environmental conditions parameters” The numerical values of the parameters are factored into the generated function in the form of input values, as described above. Further, from the parameters, the parameter values of the model 640 are derived.

The historical patient features may include, but are not limited to: a cumulative patient history of rescue events over some period of time, a cumulative count of the days that the rescue unit has been in use, the disease type, for example asthma or COPD, a record of rescue events occurring at night, and a controller medication adherence record. The patient history of rescue events may include any relevant information pertaining to the categories mentioned above for any previous rescue events. The disease type describes the severity of the patient's asthma as well as their personal treatment regimen. The controller medication adherence record contains information for the patient's use of their controller medication (which may also be associated with a sensor unit 160). This determination is evaluated by observing instances where usage was prescribed, but not performed, instances where usages was prescribed and performed, and instances where usage was not prescribed and was performed.

Current patient features may include, but are not limited to: a current latitude, longitude, and location of a coordinate of the client device 110, and the current date. The current latitude and longitude are used to determine the patient's geographical location, from which information pertaining to the patient's environmental conditions can be determined. Current rescue event data may also include a difference between the number of rescue puffs taken versus the number prescribed, as well as a count of any rescue events that may have already occurred that day and information relevant to those events. Current rescue event data may also include temporal data describing a timestamp associated with a rescue usage event, for example the month, day of the week, or the hour during the day that the rescue usage event occurred.

Air pollutant features may include information about concentrations (e.g., analog values), or more binary indications of the presence or absence of pollutants on the ground, in the atmosphere, or a combination of both. Examples of pollutants considered include, but are not limited to, carbon monoxide, ozone molecules, nitrogen dioxide molecules, sulfur dioxide molecules, particulate matter of size 2.5 micrometers or less, particulate matter of size 10 micrometers or less, and the air quality index. Specific weather features may include temperature, humidity, wind speed, wind direction, station pressure, and visibility.

Contextual parameters describe social information about a patient, their living conditions, or their environment. Examples of contextual parameters include, but are not limited to socioeconomic factors, demographic factors, built environment factors, land use factors, and behavioral health factors.

In some embodiments, contextual parameters also include social data capturing social trends and user behavior data, for example asthma searches, for a region based on trends or commonalities in patient behavior. For example, a region in which a large percentage of users perform Google searches or alternative internet-based searches related to asthma treatment is associated with a higher risk of asthma rescue usage events. As another example, a region associated with a sharp increase in inhaler rescue unit sales over a given time period may be associated with a higher risk of asthma rescue usage events. Accordingly, such social data (i.e., social trends and user behavior data) for a region at a given time may be input to the model 640 to determine a predicted number of puffs for a day occurring during that time. Some parameters, for example air pollutant parameters, weather parameters, and patient parameters, when recorded by the asthma analytics system 100, are associated with a timestamp describing the temporal details of the day during which the parameter was recorded. In one embodiment, the timestamp contains specific details describing the parameter, for example the time of day, the day, and a relation to other parameters recorded during that timestamp. In alternate implementations, each parameter may be associated with a sequential timestamp describing the time during which the parameter was recorded relative to parameters recorded during other timestamps.

Of all the aforementioned air pollutant features, weather features, and current patient features, the model 650 may select specific features to be considered when determining the risk score for a patient. Specific features may be combined into an aggregate feature which provides greater predictive value. For example, latitude and longitude data may interpreted as information describing the climate region, state, or city of the client device 110. In other embodiments, the current location of a client device 110 may be compared with a location of a point of interest (i.e., a location or area determined to be of relevance to a user or a health care provider) to determine a distance required to travel from the current location to the point of interest. The specific combination of features selected by the model 650 may be tuned during a training period or iteratively updated over periods of time.

IV.E. Long Short-Term Memory (LSTM) Network Example

Because the risk of a rescue usage event may not only be dependent on current contextual data, but rather to some extent related to previously occurring contextual data, some embodiments of the model 640 reflect such a temporal element. In one such embodiment, the model 640 is a long short-term memory (LSTM) network. Long short-term memory networks, a subclass of recurrent neural networks, can take into account the temporal nature of the data described above by using an LSTM cell(s) that processes each time step of an input data array sequentially. The LSTM cell itself contains a number of hidden layers or “gates” that interact in various ways to produce two intermediate output vectors: a hidden state and a cell state. These two intermediate output vectors, designed to persist latent information that is relevant to producing a final risk score, are inputted back into the cell along with the next time step of input data. After the last time step of input data, the final hidden state of the cell is then output to the remainder of the LSTM network, consisting of one or more layers that produce a final risk score as output.

Each layer within the network contains nodes, and each node performs a function on either the input data or the intermediate outputs of the previous layer. This function typically involves a multiplication with trainable weights and a non-linear activation function. Using some variation of gradient descent on an appropriate cost function, error may be propagated back through the network and through each time step of the cell in order to update weights accordingly after each batch sample.

An example model may include a hidden state and cell state of vector size 128, passing in 7 days of temporal data, historical contextual data, and historical patient data, with a dropout rate of 0.3 after the dense layer. The example model can be trained on a binary cross-entropy cost-function using the Adam optimizer wherein the learning rate=0.001, the beta_1=0.9, and the beta_2=0.999. In alternate embodiments, the optimal architecture for the model is periodically updated each time the model receives new temporal data. Such architecture and hyperparameter updates may be performed using Bayesian optimization tools implemented by Amazon Web Services' SageMaker product.

In embodiments in which the model 640 is an LSTM, the data analysis module 131 accesses patient data recorded over a period of time. The accessed patient data may be further sorted into individual timesteps, each of which represents a rescue inhaler usage event. The data analysis module 131 encodes the patient data, which at least includes patient parameters, weather parameters, air pollutant parameters) for each rescue inhaler usage event into a feature vector, hereafter referred to as a parameter vector. Feature values of the parameter vector each represent a value of a patient parameters, weather parameters, or air pollutant parameter recorded at the time of the rescue inhaler usage event. The time at which a rescue inhaler usage event is detected may hereafter be referred to as a “timestep.” In some embodiments, a daily or hourly timestamp may be recorded during each instance in which a rescue inhaler usage event is detected.

Because each parameter vector is an encoded representation of parameter values measured at distinct timesteps, the data analysis module 640 iteratively inputs each parameter vector to the model. The model 640 iteratively updates an intermediate output vector for each timestep occurring over the period of time based on parameter vectors encoding parameter values recorded over the timesteps and intermediate output vectors determined for previous timesteps. As an illustrative example, at a first timestep T1, the model 640 receives the parameter vector P1 representing parameter values recorded at T1 as an input and outputs an intermediate output vector I1. At a second timestep T2, the model 640 receives the parameter vector P2 representing parameter values recorded at T2 and the intermediate output vector I1 as inputs and outputs an updated intermediate output vector 12. Accordingly, the model 640 iteratively receives parameter vectors for timesteps preceding a current timestep to generate a final output vector that provides a latent representation of the sequence of parameter values associated with rescue inhaler usage events occurring over the period of time leading up to the current timestep (e.g., a current output vector). The current output vector is input to the personalized risk module 650 to determine a risk score for the patient at the current timestep.

IV.F. Asthma Rescue Event Risk Notifications

The notification module 645 generates 435 a risk score notification including any one or more of: the categorization, the patient-specific risk score, the predicted number of puffs, potential causes for the risk score and the predicted number of puffs, and options that the patient can take to prevent the occurrence of another rescue usage event under these circumstances. As discussed above, the application server 130 generates 435 a risk score notification for one or more of: the patient 111, their healthcare provider 112, and/or any other authorized individuals.

The risk notification may include a wide variety of informational content, including the risk score, the categorization, and any of the input values from any of the parameters of the model 640.

The risk notification may also be comprised of a recommendation regarding how to prevent future rescue inhaler events based on the parameters responsible for the change in the risk categorization. For example, the recommendation may include following the adherence schedule more closely, increasing the dose or usage of a controller medication, avoiding that geographical region altogether, or limiting exposure to areas with similar environmental conditions.

Additionally, a patient 111 may be provided a geographical map with pinpoints of all of their medication rescue event locations that have occurred in the last year. As another example, a patient 111 may be provided with a geographical map include where event 410 took place, along with pinpoints of all medication rescue events that have occurred in the nearby geographical area either that day or within a threshold period of time, to indicate recent patterns in medication rescue events for that area. As another example, the healthcare provider 112 of the patient 111 may be presented with medication rescue event data from their patients 111 from that day or a recent period of time to help the provider 112 identify medication rescue event trends.

The risk notification may also be delivered in many other situations which depending upon the implementation may be based on triggering conditions, or may be sent to the client device according to some other mechanism. For example, if a patient's current condition worsens due to changes in patient parameters (e.g., decreased patient controller medication adherence trend) or environmental condition parameters (e.g., low concentrations of ozone, greater amounts of pollutants in the air, or higher humidity) an updated risk notification is delivered to the patient 111. As described above, in another implementation, a risk notification may be presented to a patient when their patient-specific risk score increases or when their risk score is re-categorized, for example from “fair” to “poor.” In additional implementations, notifications of patient-specific risk scores or categorizations may be presented to medical providers to inform staffing, medical supplies, or to describe the medical condition for a clinic or group of patients. Alternatively, if a patient's current condition improves due to similar changes, an updated risk notification may also be delivered. Risk notifications may also be delivered at a patient's request, for example due to a verbal request for local asthma conditions from a third party device (e.g., Google Home™ or Amazon Alexa™).

As described above, generally risk notifications are delivered through the client device 110, however in other embodiments, in the event of improved or worsened conditions, risk notifications may be delivered as an SMS notification, an email notification, a notification from an embeddable widget with local asthma conditions, or notifications from various IFTTT applets (https://iflll.com/).

### V. EXAMPLES

### V.A. Example 1

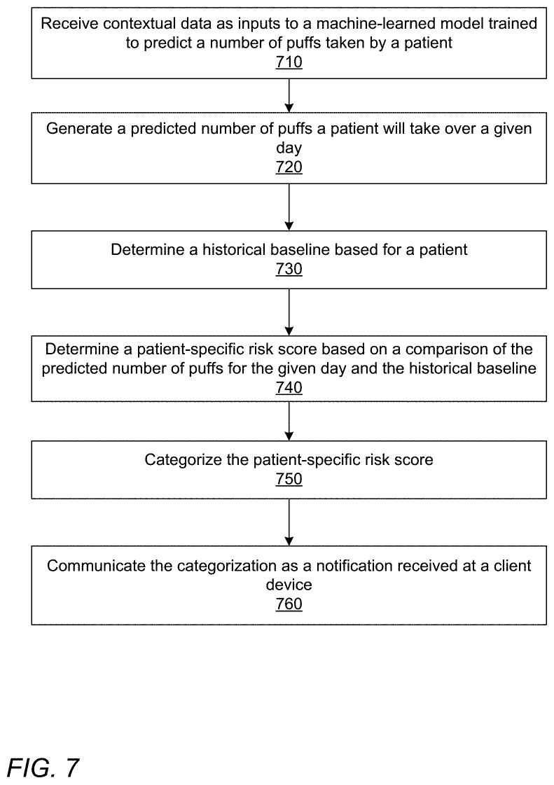

FIG. 7A illustrates an example receiver operating characteristics curve on an unseen, forward-looking test set. The baseline model 650 implementing an LSTM network achieved an area under the receiver operating characteristic curve of 0.90, thereby affirming the efficacy of the LSTM-implemented model.

### V.B. Example 2

FIG. 7B illustrates an example precision-recall curve on an unseen, forward-looking test set. The baseline model 650 implementing an LSTM network achieved an area under the precision-recall curve of 0.72, thereby affirming the efficacy of the LSTM-implemented model.

### VI. BENEFITS

The asthma rescue event risk notifications provided to patients 111 and providers 112 convey many benefits. Patients are informed of their risk of an asthma rescue event in real time or near real time (on the order of seconds to minutes), and can take action to prevent that occurrence, for example by improving their adherence to their controller medication, staying away from geographic areas with adverse conditions (e.g., air pollution concentrations), adding or altering their prescribed medication regimen (such as an adjustment of dosage or the introduction of antibiotics or systemic corticosteroids), or scheduling an appointment with their doctor to address their recent spike in use of rescue medication which in turn would reduce the frequency of emergency room and hospital visits for the patient. Because the event data is automatically reported to the application server 130 without the need for patient input, the accuracy and quality of the event data is improved relative to manually-collected data by a health care provider 112 or other entity, and thus the accuracy of the conclusion for the risk of asthma rescue events is also improved.

Additionally, follow up notifications provided by the application server 130 including reports of nearby rescue medication events by other patients can provide patients with additional information about others who are suffering from similar issues and provide regional information relevant for the patient's decision-making. Follow up notifications can further encourage the user regarding progress on taking their adherence medication. Ideally such notifications will prevent the occurrence of asthma rescue events, thus preventing patient harm as well as hospital visits and their associated costs.

Health care providers informed of the risk of one or more of their patients' risk of asthma rescue events can similarly track the progress of their patients, and use the information to update their treatment regimen for each patient, schedule appointments with patients, and so on. For patients at high risk of an asthma rescue events, the notification may be a call to action for the health care provider to communicate with the patient and encourage them to seek medical treatment. Follow up notifications may provide information about regional effects (e.g., pollution), patient controller medication adherence, etc.

### VII. ADDITIONAL CONSIDERATIONS

Although the discussion above focuses on asthma specifically, all systems and processes described herein are equally applicable to chronic obstructive pulmonary disease (COPD) and chronic respiratory disease (CRD) generally, and consequently can also be used to assist in treatment of COPD and CRD, as well as asthma.

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A method comprising:
accessing, for a patient, a sequence of parameter vectors corresponding to timesteps occurring over a period of time, wherein each parameter vector of the sequence comprises a set of values describing at least one patient parameter, weather parameter, and air pollutant parameter recorded by a medicament device sensor during a rescue inhaler usage event, the medicament device sensor removably attached to a rescue inhaler unit and configured to monitor medicament usage of the rescue inhaler unit when the rescue inhaler unit dispenses a rescue medication to the patient;
generating a current output vector providing a latent representation of the sequence of parameter values associated with rescue inhaler usage events occurring over the period of time by inputting the sequence of parameter vectors to a machine-learned model, wherein the machine-learned model is trained to:
iteratively update an output vector for each timestep occurring over the period of time based on a corresponding parameter vector to generate the current output vector at a current timestamp based on the parameter vector for the current timestep an output vector computed for a timestep immediately preceding the current timestep; and
determine a predicted number of rescue inhaler usage events for the current timestep based on the current output vector;
inputting the predicted number of medication usage events to a function to determine a risk score for the current timestep; and
sending risk notification to a client device operated by the patient describing the risk score determined for the current timestep.

2. The method of claim 1, further comprising:
determining a historical baseline based on a daily record of medication usage by the patient over a preceding period of time;
inputting the predicted number of rescue inhaler usage events to the function, wherein the function compares the predicted number of rescue inhaler usage events for the current timestep to the historical baseline; and
determining the risk score for the current timestep based on the comparison.

3. The method of claim 1, further comprising:
determining a historical baseline based a standard deviation of recorded rescue inhaler usage events occurring a preceding period of time;
inputting the predicted number of rescue inhaler usage events to the function, wherein the function compares the predicted number of rescue inhaler usage events for the current timestep to the historical baseline; and
determining the risk score for the current timestep based on the comparison.

4. The method of claim 1, further comprising:
receiving a record of rescue inhaler usage events occurring over the period of time from the client device, medicament device sensor, or rescue inhaler unit.

5. The method of claim 1, further comprising:
inputting the current output vector into the function to determine a risk score for the current timestep in response to a triggering condition, wherein the triggering condition is one or more of the following:
a change in a physical location of the client device;
a conclusion of the period of time;
change in a value of at least one patient parameter, weather parameter, and air pollutant parameter; and
occurrence of a rescue inhaler usage event during the current timestep detected when the rescue inhaler unit dispenses rescue medication to the patient during the current timestep.

6. The method of claim 1, wherein the machine-learned model is a long short-term memory neural network.

7. The method of claim 1, wherein the risk score is a numerical value representing a likelihood that the patient experiences a rescue inhaler usage event during the current timestep.

8. The method of claim 1, wherein the set of values further comprise at least one event data parameter and at least one contextual data parameter.

9. The method of claim 1, wherein the set of values comprise one or more of the following:
at least one historical patient parameter, further comprising:
a cumulative patient history of rescue events;
a patient history of events occurring at night;
a cumulative count of days using the rescue unit;
a disease type; and
an adherence record.

10. The method of claim 1, wherein the set of values comprise one or more of the following:
at least one current patient parameter, further comprising:
a current latitude and longitude coordinate of the client device;
a current location;
a current date; and
a difference in number of rescue puffs taken and prescribed for the rescue event.

11. The method of claim 1, wherein the set of values comprise one or more of the following:
at least one air pollutant parameter, further comprising:
ozone molecules (O₃);
carbon monoxide molecules (CO);
nitrogen dioxide molecules (NO₂);
sulfur dioxide molecules (SO₂);
particulate matter, 2.5 micrometers or less (PM_{2.5});
particulate matter, 1 micrometer or less (PM_{1.0}); and
air quality index (AQI).

12. The method of claim 1, wherein the set of values comprise one or more of the following:
at least one weather parameter, further comprising:
temperature;
humidity;
wind speed;
wind direction;
station pressure; and
visibility.

13. The method of claim 1, wherein the set of values comprise one or more of the following:
social parameters for a geographic region; and
behavioral parameters for a geographic region.

14. The method of claim 1, wherein the set of values comprise a number of days where rescue inhaler usage events are monitored by a computing system external to the rescue inhaler unit.

15. The method of claim 1, wherein the function determines whether a number of rescue inhaler usage events for a given day exceeds a threshold.

16. The method of claim 1, wherein the risk notification is presented at a timestep following the current timestep at which the risk score is determined.

17. The method of claim 1, wherein the risk notification comprises informational content regarding a triggering condition.

18. The method of claim 1, wherein the risk notification comprises informational content
regarding at least one of:
a record of rescue inhaler usage events occurring over the period of time;
a threshold against which the risk score was compared; and
a risk categorization based on the risk score;

19. The method of claim 1, wherein the risk notification further comprises informational content regarding the rescue inhaler usage event, wherein the informational content further comprises a subset of patient parameters, weather parameters, and air pollutant parameters responsible for a change in risk categorization compared to a previous period of time.

20. The method of claim 1, wherein the risk notification further comprises informational content regarding the rescue inhaler usage event, wherein the informational content further comprises a recommendation regarding how to prevent future rescue inhaler usage events based on the subset of patient parameters, weather parameters, and air pollutant parameters responsible for the change in risk categorization.

21. A non-transitory computer readable storage medium comprising computer program instructions that when executed by a computer processor cause the processor to:
access, for a patient, a sequence of parameter vectors corresponding to timesteps occurring over a period of time, wherein each parameter vector of the sequence comprises a set of values describing at least one patient parameter, weather parameter, and air pollutant parameter recorded by a medicament device sensor during a rescue inhaler usage event, the medicament device sensor removably attached to a rescue inhaler unit and configured to monitor medicament usage of the rescue inhaler unit when the rescue inhaler unit dispenses a rescue medication to the patient;
generate a current output vector providing a latent representation of the sequence of parameter values associated with rescue inhaler usage events occurring over the period of time by inputting the sequence of parameter vectors to a machine-learned model, wherein the machine-learned model is trained to:
iteratively update an output vector for each timestep occurring over the period of time based on a corresponding parameter vector to generate the current output vector at a current timestamp based on the parameter vector for the current timestep an output vector computed for a timestep immediately preceding the current timestep; and
determine a predicted number of rescue inhaler usage events for the current timestep based on the current output vector;
input the predicted number of medication usage events to a function to determine a risk score for the current timestep; and
sending a notification to a client device operated by the patient describing the risk score determined for the current.
